(19) United States
(12) Patent Application Publication
(10) Pub. No.: US 2026/0153472 A1
(43) Pub. Date: Jun. 4, 2026

(54) SENSOR ELECTRODE FOR HALOGEN DETERMINATION AND METHOD FOR MANUFACTURING A SENSOR ELECTRODE

(71) Applicant: Analytik Jena GmbH+Co. KG, Jena (DE)

(72) Inventors: Marcel Freundel, Ilmenau (DE); Heiko Henneberg, Plaue (DE); Henri-Alan Traute, Ilmenau (DE); Sophia Kaufmann, Rudolstadt OT Teichel (DE); Christiane Ehrling, Ilmenau (DE)

(21) Appl. No.: 19/408,564

(22) Filed: Dec. 4, 2025

(30) Foreign Application Priority Data
Dec. 4, 2024 (DE) 10 2024 136 105.0

(51) Int. Cl. G01N 27/30; G01N 27/42; G01N 33/00

(52) U.S. Cl. CPC G01N 27/30; G01N 27/423; G01N 33/0052

(57) ABSTRACT

A sensor electrode for halogen determination includes an electrode shaft with two opposite end faces, at least two electrode pins, two elongated plate-like inserts, and a connection component. The electrode pins project beyond or terminate flush with a first end face of the shaft, and at least one pin is electrically and mechanically connected to one of the inserts. The inserts are arranged within the shaft, and at least one insert is electrically and mechanically connected to the connection component located on the second end face. The electrode shaft is at least partially formed by an injection molding process, and the electrode pins are at least partially embedded in the shaft.

### CROSS-REFERENCE TO RELATED APPLICATION

The present application is related to and claims the priority benefit of foreign patent application No DE 10 2024 136 105.0, filed on Dec. 4, 2024, the entire contents of which are incorporated herein by reference.

### TECHNICAL FIELD

The present disclosure relates to a sensor electrode for halogen determination. Furthermore, the present disclosure relates to a method for manufacturing a sensor electrode for halogen determination.

### BACKGROUND

In the prior art, it is known to use biamperometry for the determination of traces of halogens (other than fluorine, i.e., chlorine, bromine or iodine). This is a variant of amperometry in which two identical working electrodes are used. An electrolysis current only flows when a material conversion takes place at both working electrodes. The halogen content is then determined from the conductivity of the electrolyte. A typical application is AOX analysis according to ISO standard 9562. Organochlorine compounds are adsorbed on activated carbon. The samples prepared in this way are burned in an oxygen stream in a furnace and the resulting HCl (HX) is quantified in a coulometric measuring cell after sulfuric acid drying.

DE 42 31 960 A1 discloses a sensor electrode in which two electrode pins are combined as working electrodes in one component. The electrical connection components of the electrode pins are arranged in an electrode shaft made of ceramic. The electrode pins protrude beyond the end face of the electrode shaft or are flush with the end face in order to come into contact with the electrolyte for the determination of the chloride content.

With such a sensor electrode, it is important for the accuracy and precision of the measurement results that the effective surface of the electrode pins and the distance between them are the same for sensor electrodes. It is also important to ensure that the electrode pins in the ceramic shaft are well sealed. Both requirements mean that automation of the production of the sensor electrodes is hardly possible and cost-intensive manual work must be used.

DE 10 2008 054 659 A1 describes the production of conductive conductivity sensors by an injection molding method. DE 10 2018 121 787 A1 discloses an electrode assembly for an amperometric sensor, the electrode body of which is produced by an injection molding method. DE 10 2017 220 847 A1 describes a conductivity sensor that is produced by a plastics material injection molding method, whereby the electrodes are embedded. DE 10 2021 112 181 A1 discloses a sensor whose conductor tracks (e.g., made of silver) can be overmolded. DE 10 2016 110 856 A1 indicates that an electrode assembly is manufactured in the form of an injection-molded formwork support. An electrode arrangement is also described in DE 20 2011 101 241 U1. An adapter for colloid electrodes can be found in DE 20 2017 006 380 U1.

### SUMMARY

The object of the present disclosure is to propose a sensor electrode for halogen determination which can be manufactured as easily as possible without compromising the quality of the measurements. Furthermore, the task is to propose a manufacturing process for a sensor electrode that is as simple and reliable as possible.

The object is achieved by a sensor electrode for halogen determination, wherein the sensor electrode has at least two electrode pins, two elongated, plate-like inserts, a connection component and an electrode shaft, wherein the electrode shaft has two mutually opposite end faces, wherein the electrode pins are arranged such that the electrode pins project beyond the one end face of the electrode shaft or terminate flush with the end face, wherein at least one electrode pin is electrically and mechanically connected to at least one of the two inserts, wherein the two inserts are arranged in the electrode shaft, wherein the connection component is arranged on the other end face of the electrode shaft, wherein at least one of the two inserts is electrically and mechanically connected to the connection component, wherein the electrode shaft is at least partially produced by an injection molding method, and wherein the electrode pins are at least partially embedded in the electrode shaft.

The sensor electrode according to the present disclosure has at least two electrode pins, two elongated, plate-like inserts, a connection component and an electrode shaft. The inserts are elongated and plate-like. In one variant, the inserts are at least partially made of circuit board material. The inserts are, on the one hand, a basic mechanical structure of the electrode and, on the other hand, they also serve to electrically contact the electrode pins.

The electrode shaft is e.g., substantially cylindrical or circular-cylindrical. It has an end side from which the electrode pins protrude or with which they terminate flush. For electrical measurements, the electrode pins are connected to the inserts located in the electrode shaft. The inserts are guided through the electrode shaft, are held by the shaft and are thereby also protected from the environment. The electrode shaft is at least partially or completely produced by an injection molding method. The injection molding method allows components to be embedded directly in the electrode shaft and thus sealed. The inserts are also securely protected against tension in the electrode shaft. In addition, special geometries of the electrode shaft can also be created through injection molding. Furthermore, the electrode shaft has another end face, which can also be referred to as the neck or neck end face or as the connection end face, which faces away from the process and serves e.g., to fix the electrode shaft in a wall, etc.

Overall, sensor electrodes with reproducible properties can be produced cost-effectively. The material for injection molding may be electrically insulating. For example, such a material is used or it is processed in such a way that the electrode shaft has the smoothest possible outer surface, which is easy to clean and minimizes carryover. The partial embedding of the electrode pins in the electrode shaft results in good sealing, precise positioning of the pins and very good mechanical stability. The connection component allows the electrode to be connected to cables and therefore also to other devices.

In at least one embodiment an electrode pin is designed as a cathode pin and serves as a generator cathode. The sensor electrode is, for example, used in a coulometric measuring cell which consists of the two sensor electrodes (for example, made of silver) and a pair of generator electrodes. In the case of coulometric chloride titration, the generator system consists of a platinum cathode and a silver anode. The generator cathode (for example, made of platinum) is integrated with the cathode pin in the sensor electrode. This is advantageous because the volume of the measuring cell is small and the electrodes can thus be arranged in a space-saving manner.

In at least one embodiment of the sensor electrode, the electrode shaft consists at least partially of a plastics material. In such embodiments, an injection molding method is carried out with a plastics material. The plastics material may be polypropylene. For example, the plastics material is resistant to chemicals, including concentrated acetic and sulfuric acid. For example, the plastics material is polyvinylidene fluoride (PVDF), which includes hardness, impermeability and resistance to chemicals. The plastics material may be able to be sprayed. Possible materials are thus for example thermoplastics, thermosets or elastomers. When selecting the plastics material, an important factor is its resistance, which refers both to the interior, i.e., in relation to the overmolded components (i.e., primarily thermal resistance), and to the exterior when using the sensor electrode in relation to any chemicals that may be present (i.e., primarily chemical resistance). The plastics material may embodied such that it neither swells nor shrinks during use of the sensor electrode in order to ensure tightness.

At least one embodiment of the sensor electrode includes two electrode pins comprising silver. The sensor electrode with the silver electrode pins is used in part for the determination of halogens, for example, chloride. The injection molding method allows the electrode pins to be tightly enclosed. This is in contrast to the difficulties in the prior art, which, for example, does not allow silver pins to be cast in glass.

In a further embodiment, two electrode pins consist at least partially of platinum. The associated sensor electrode can be used for example for the biamperometric determination of SO2.

At least one embodiment of the sensor electrode includes that the sensor electrode further comprises a cathode pin. This, for example, consists at least partly of platinum.

At least one embodiment includes that the two inserts have electrical conduction structures, that the two electrode pins, which consist at least partially of silver, are connected to the electrical conduction structures of the one insert, and that the electrode pin, which consists at least partially of platinum, is connected directly or indirectly to the electrical conduction structures of the other insert. In this embodiment, the total of three electrode pins are distributed between the two inserts by connecting two silver electrode pins to the conduction structures of one insert and one platinum electrode pin to the conduction structures of the other insert. In at least one of the following embodiments, it is provided that the platinum electrode pin is indirectly connected to the conduction structures of the other insert via an adapter sleeve.

In at least one further embodiment, another electrode pin serves as a generator anode. This embodiment is, for example, associated with the sensor electrode having four electrode pins. The result is a compact 4-electrode system designed as one assembly.

At least one embodiment of the sensor electrode is that the connection component allows a reversible electrical connection of the sensor electrode with a plug. In these embodiments, the sensor electrode can be reversibly connected to a plug. This simplifies for example the replacement or cleaning of the sensor electrode. For example, a control or evaluation circuit is connected to the connector.

At least one embodiment of the sensor electrode includes that the electrode shaft further comprises at least one receiving groove for receiving a sealing component. The sealing component is, for example, an O-ring that is inserted into the receiving groove. The sealing component seals, for example, the transition between the electrode shaft and a part of a measuring cell in which the reaction liquid or electrolyte to be measured is located.

At least one embodiment provides that at least one of the two inserts has electrical conduction structures, and that the inserts are designed and arranged relative to one another in such a way that planes in which the inserts mainly extend are substantially perpendicular to one another. In this embodiment, two inserts are present, each extending mainly in the longitudinal direction and each being designed for example partly as a printed circuit board or from printed circuit board material. The two inserts here should be perpendicular to each other so that they form a secure base structure for the elongated electrode shaft. At least one insert has an electrical conduction structure to which at least one electrode pin can be connected. In at least one embodiment, each of the two inserts has a conduction structure.

The following embodiments relate to the inserts and how they are arranged relative to each other, e.g., the overall geometric structure that they form. It should be remembered that on the one hand the inserts form the basic structure of the electrode shaft and on the other hand serve to electrically connect the electrode pins to the connection component. Ultimately, the outer extension of the electrode shaft is determined by the inserts. The geometry of the inserts may be selected such that the insert(s) withstand injection molding during the manufacture of the electrode shaft.

At least one embodiment includes that the inserts are designed and arranged relative to one another in such a way that the inserts together substantially form a T-shape. T-shape here refers to the capital letter T. In at least one embodiment, its crossbeam is slightly pierced by its longitudinal beam.

At least one embodiment provides that the inserts are designed and arranged relative to one another in such a way that the inserts together substantially have the shape of a plus sign. In these embodiments, the two inserts form an X, cross, or plus sign shape. In at least one embodiment, the four components of the plus sign have substantially the same length. The shape of the inserts is shown in each case in a section perpendicular to their longitudinal axis.

In at least one alternative embodiment, there is one insert, which has either a T-shape or a plus sign shape. Thus, in at least one embodiment there is a central support structure, at one end of which another transverse substructure is attached (i.e., a T-shape). Alternatively, two support structures cross each other, for example, in the area of their centers, including forming right angles with each other (i.e., a plus sign shape). As an alternative to the latter variant, the two support structures can intersect at an obtuse or acute angle (i.e., a cross or a St. Andrew's cross shape).

At least one embodiment includes that at least one of the two inserts has electrical conduction structures, that the inserts are designed and arranged relative to one another in such a way that one insert at least partially protrudes through the other insert. In these embodiments, the two inserts are connected to each other by one insert protruding through the other. This results in a basic mechanical stability that is relatively easy to achieve. The embodiment is associated with the fact that one insert is designed in such a way that the other insert can be passed through it.

At least one embodiment includes that the at least two inserts are connected to each other via soldering points. The soldering points also serve to stiffen the two inserts. In at least one embodiment, soldering points are present along the entire length where the two inserts are in direct contact with each other.

In at least one alternative or additional embodiment, the at least two inserts are connected to each other via adhesive dots. In these embodiments an adhesive is thus used.

At least one embodiment provides that at least one electrode pin, or two electrode pins, are connected by a soldered connection to the electrical conduction structures of at least one of the two inserts.

At least one embodiment includes that at least one electrode pin is resiliently arranged in an adapter sleeve and is electrically connected to the adapter sleeve, and that the adapter sleeve is electrically connected to the electrical conduction structures of the at least one of the two inserts. These embodiments take into account the fact that some types of electrode pins cannot be connected to other components (here to at least one of the two inserts) by means of a solder connection, or can be so connected with considerable effort. This applies, for example, to the material platinum. Therefore, in the embodiment an adapter sleeve is provided which enables at least mechanical stabilization and basic electrical contacting. The adapter sleeve is designed in such a way that it enables, for example, a soldered connection with the conductor structures of at least one of the two inserts. For this purpose, the adapter sleeve has, for example, a spring-loaded contact which makes electrical contact with the electrode pin.

At least one embodiment provides that the electrode pin arranged in the adapter sleeve is inserted into the adapter sleeve and is fixed and sealed as a result of the production of the electrode shaft by the injection molding method. In these embodiments, the injection molding method used to produce the electrode shaft also seals the region of the electrode pin and the adapter sleeve.

At least one embodiment includes that the connection component is designed as a multi-pin socket in the direction of the inserts. At least one embodiment is, for example, a three-pin socket to which three electrode pins are connected indirectly via the inserts.

At least one embodiment provides that the electrode shaft and the connection component are designed and arranged such that a cross section of the electrode shaft along the electrode shaft is substantially symmetrical. In this embodiment, the inserts are designed and realized in such a way that the cross section through the electrode shaft is symmetrical. This is intended to prevent material accumulation during the production of the electrode shaft. Such accumulations often lead to delays and should therefore be avoided. For example, it may desirable to ensure that the individual components of the sensor electrode within a mold, which may be used in the injection molding method, have constant distances from the inner surface of the mold. This makes it possible to achieve an substantially straight and completely closed coating of the inner parts of the electrode shaft. In addition, this avoids asymmetrically acting forces during the injection molding method.

Furthermore, the object is achieved by a method for producing a sensor electrode for halogen determination, the method comprising at least the following steps: that multiple electrode pins are arranged relative to one another, that at least one electrode pin is electrically and mechanically connected to one of at least two inserts, that an electrode shaft is produced around the electrode pins and around the two inserts at least partially using an injection molding method such that the electrode pins and the inserts are at least partially embedded in the electrode shaft and that at least one electrode pin partially projects beyond an end face of the electrode shaft, and that after the electrode shaft has been produced, the at least one electrode pin projecting beyond the end face is changed in the degree by which it projects beyond the end face.

The statements and explanations regarding the sensor electrode also apply to the method and vice versa, so that repetition is omitted. The sensor electrode may be an electrode manufactured by the method.

According to the present disclosure, the two electrode pins are arranged to fit each other (e.g., in a mold) in order to subsequently produce the electrode shaft by injection molding. At least one electrode pin is connected to one of the two inserts and the electrode shaft is produced around the pins and the inserts. The electrode pins are inserted into the electrode shaft with one side and protrude beyond it with the other side or terminate flush with the surface in order to come into contact with the medium whose chloride content is to be ascertained. Furthermore, after the electrode shaft has been produced, at least one electrode pin is machined in terms of its dimensions and thus in relation to the reaction surface in such a way that, for example, a desired measurement accuracy is achieved.

In at least one embodiment, two inserts are inserted into each other. Two electrode pins are then soldered to the conduction structures of at least one insert. An electrode pin is inserted into an adapter sleeve, which in turn is also electrically contacted, e.g., soldered, with the conductor structures of the inserts. The electrical connections are electrically insulated from each other. The fixing of the arrangement is done in a tool using the protruding elements, e.g., the electrode pins. The electrode shaft is then produced using the injection molding method.

### BRIEF DESCRIPTION OF THE DRAWINGS

The present disclosure is explained in greater detail with reference to the following figures;

FIG. 1 shows a section through a schematic measuring arrangement for the determination of the chloride content;

FIG. 2 shows the components of a sensor electrode in an exploded view;

FIG. 3 shows a part of the sensor electrode of FIG. 2 in the assembled state;

FIG. 4 shows the components of FIG. 2 in the mounted state;

FIG. 5 shows a variant of the components of a sensor electrode in the mounted state; and

FIG. 6 shows a spatial representation of a section through the components of FIG. 5.

### DETAILED DESCRIPTION

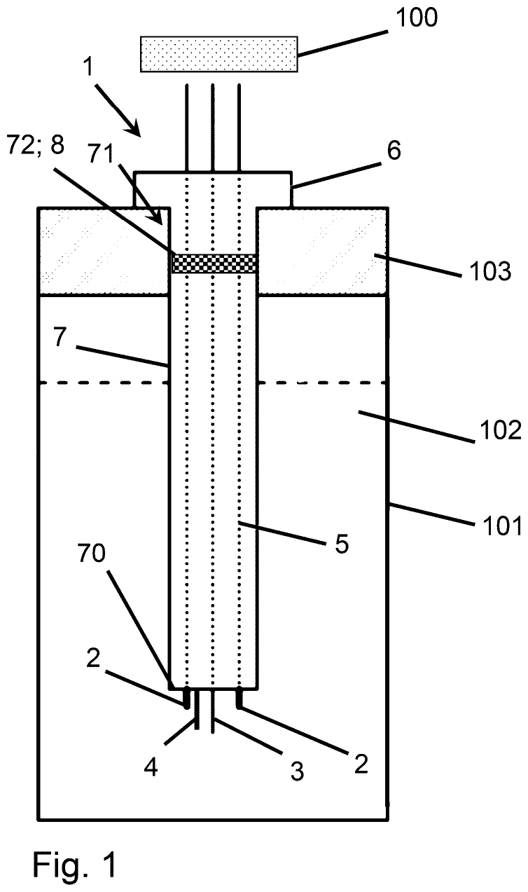

FIG. 1 shows a sensor electrode 1 located in a measuring cell 101. The measuring cell 101 is closed on one side with a measuring cell cover 103, wherein the sensor electrode 1 protrudes through a continuous recess of the measuring cell cover 103. A sealing component 8 arranged in a receiving groove 72 of the electrode shaft 7, which component is here for example an O-ring, seals the transition between the electrode shaft 7 and the recess of the measuring cell cover 103.

The reaction liquid 102 is located in the measuring cell 101, into which the two identically designed electrode pins 2, which may be made of silver, protrude and provide the measurement.

The electrode shaft 7 is substantially circular-cylindrical and has at its upper end a connection component 6 with a larger outer diameter than the inner diameter of the recess in the measuring cell cover 103. This holds the electrode shaft 7 in the measuring cell cover 103. The connection component 6 is further designed such that the sensor electrode 1 can be reversibly electrically contacted by a plug 100.

Opposite the connection component 6, the two electrode pins 2, which may be made of silver, are arranged in the end face 70 of the electrode shaft 7 and are embedded there. Therefore they are well insulated. For the measurement, an electrode pin is further provided as a cathode pin 3, which also projects beyond the end face 70, is embedded in the end face 70 and is electrically contacted with at least one insert 5. The cathode pin 3 serves as the generator cathode. In the measurement, the fourth electrode pin 4 performs the function of the generator anode. The insert 5 provides the electrical connections between the electrode pins 2, 3, 4 and the connection component 6 and forms the geometric basic structure of the circular-cylindrical electrode shaft 7.

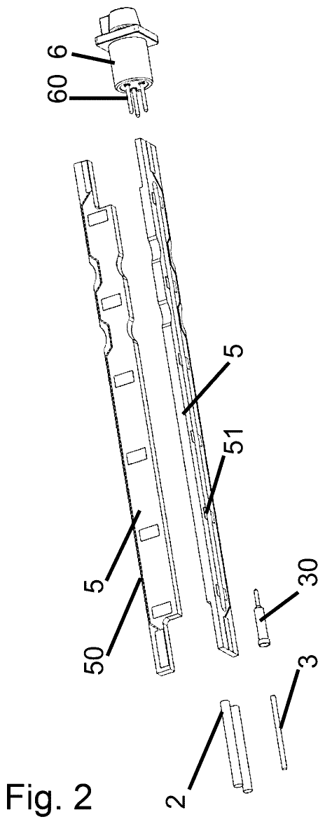

FIG. 2 shows that the majority of the spatial extent of the sensor electrode 1 results from the shape of the (here) two inserts 5. The two inserts 5 are made of circuit board material and extend mainly in the longitudinal direction, i.e., along the longitudinal axis of the sensor electrode 1. The inserts 5 have conduction structures 50 which serve to electrically contact the electrode pins 2, 3 and the connection component 6.

One insert 5 has a recess 51 into which a part of the other insert 5 is to be inserted. This results in the basic mechanical structure of the electrode shaft 7.

At the upper (see FIG. 1), or here right, end is located the connection component 6, which in the application of the sensor electrode 1 can be connected to a plug 100 and therefore to a device for the measurement. In the exemplary embodiment, the connection component 6 has three poles 60 which are connected to the three electrode pins 2, 3.

For the measurements, there are three electrode pins 2, 3, of which two electrode pins 2 are made of silver and one electrode pin 3 is made of platinum. The silver electrode pins 2 are connected to the conductive structures 50 by soldering. The electrode pin 3 made of platinum is inserted into the adapter sleeve 30 in order to be spring-mounted and electrically contacted there. The sealing of the electrode pin 3 in the adapter sleeve 30 is done, as with the other electrode pins 3 2, via the injection molding method by which the electrode shaft 7 is produced.

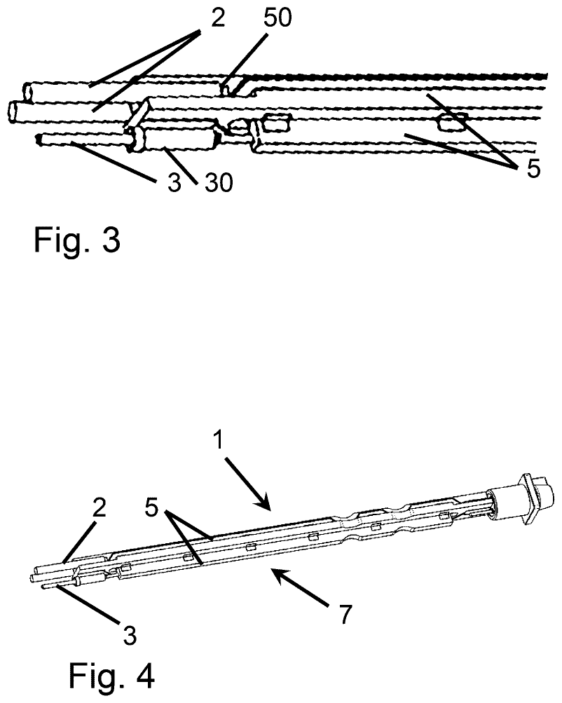

FIG. 3 shows the front end of the sensor electrode 1 with the three electrode pins 2, 3. The two silver electrode pins 2 are directly connected to the conductive structure 50 of one insert 5. The platinum electrode pin 3 is inserted into the adapter sleeve 30, which is electrically and mechanically contacted with the conductor structure 50 of the other insert 5. The cable structure 50 can be arranged at least partially within or on the corresponding insert 5.

FIG. 4 shows the assembled components of the electrode shaft 7 to be overmolded. The electrode pins 2, 3 protrude at the front and are sealed by the injected plastics material. It can be seen clearly that the two inserts protrude through each other and are therefore perpendicular to each other overall.

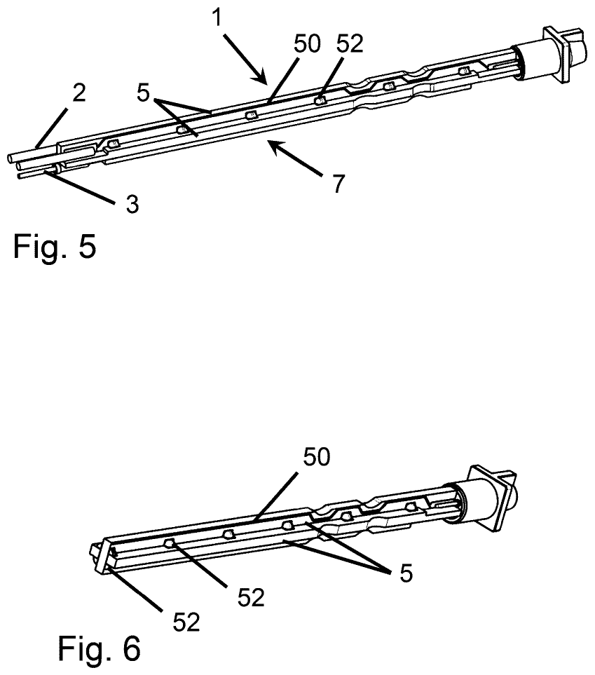

FIG. 5 shows how, in the embodiment shown, a total of six soldering points 52 are situated in a row in the angle between the two inserts 5. The soldering points 52 connect the two inserts 5 to each other and stiffen the arrangement of the two inserts 5. The distance between the soldering points 52 in the tip of the angle enclosed by the two inserts 5 and the conduction structure 50 further out on the insert 5 can be seen. It can be seen below the horizontally running insert 5 that there is at least one further row of soldering points 52.

The sectioned view of FIG. 6 shows that there is a row of soldering points 52 in each of the four intersection angles between the two inserts 5 and thus within the four corners. In this case, each row consists of six soldering points 52, so that the two inserts 5 are connected to each other by a total of 24 soldering points. FIG. 6 also illustrates how the insert 5, which is arranged vertically here, protrudes centrally through the insert 5, which is arranged horizontally here, so that a cross shape results.

1. A sensor electrode for halogen determination comprising:
an electrode shaft having two opposite end faces;
at least two electrode pins, each electrode pin projecting beyond or terminating flush with a first end face of the electrode shaft;
two elongated plate-like inserts arranged in the electrode shaft; and
a connection component arranged on a second end face of the electrode shaft;
wherein at least one of the electrode pins is electrically and mechanically connected to at least one of the two inserts;
wherein at least one of the two inserts is electrically and mechanically connected to the connection component,
wherein the electrode shaft is at least partially formed by an injection molding process, and
wherein the electrode pins are at least partially embedded in the electrode shaft.

2. The sensor electrode according to claim 1,
wherein the electrode shaft comprises a plastics material.

3. The sensor electrode according to claim 1,
wherein the at least two electrode pins comprise silver.

4. The sensor electrode according to claim 1,
wherein the at least two electrode pins comprise platinum.

5. The sensor electrode according to claim 1, further comprising an electrode pin configured as a cathode pin.

6. The sensor electrode according to claim 1, wherein:
the two inserts include electrical conduction structures;
at least one electrode pin comprising silver is connected to the electrical conduction structures of one of the two inserts; and
at least one electrode pin comprising platinum is connected directly or indirectly to the electrical conduction structures of the other insert.

7. The sensor electrode according to claim 1,
wherein at least one electrode pin is configured as a generator anode.

8. The sensor electrode according to claim 1,
wherein the connection component is configured to allow a reversible electrical connection to a plug.

9. The sensor electrode according to claim 1,
wherein the electrode shaft further comprises at least one receiving groove configured to receive a sealing component.

10. The sensor electrode according to claim 1,
wherein at least one of the two inserts includes electrical conduction structures, and
wherein the inserts are embodied and arranged relative to one another such that planes in which the inserts primarily extend are substantially perpendicular to one another.

11. The sensor electrode according to claim 10,
wherein the inserts are embodied and arranged relative to one another such that the inserts together substantially form a T-shape.

12. The sensor electrode according to claim 10,
wherein the inserts are embodied and arranged relative to one another such that the inserts together substantially form a plus sign shape.

13. The sensor electrode according to claim 1,
wherein at least one of the two inserts includes electrical conduction structures, and
wherein the inserts are embodied and arranged relative to one another such that one insert at least partially projects through the other insert.

14. The sensor electrode according to claim 13,
wherein the at least two inserts are connected to one another via soldering points.

15. The sensor electrode according to claim 1,
wherein at least one electrode pin is connected by a soldered connection to the electrical conduction structures of the at least one of the two inserts.

16. The sensor electrode according to claim 1,
wherein at least one electrode pin is resiliently arranged in an adapter sleeve and electrically connected to the adapter sleeve, and
wherein the adapter sleeve is electrically connected to the electrical conduction structures of the at least one of the two inserts.

17. The sensor electrode according to claim 16,
wherein the electrode pin arranged in the adapter sleeve is inserted into the adapter sleeve and is fixed and sealed during formation of the electrode shaft by the injection molding process.

18. The sensor electrode according to claim 1,
wherein the connection component is embodied as a multi-pole socket oriented toward the inserts.

19. The sensor electrode according to claim 1,
wherein the electrode shaft and the connection component are embodied and arranged such that a cross section of the electrode shaft along its length is substantially symmetrical.

20. A method for manufacturing a sensor electrode for halogen determination, the method comprising:
arranging a plurality of electrode pins relative to one another;
electrically and mechanically connecting at least one of the electrode pins to one of at least two inserts;
forming an electrode shaft at least partially by an injection molding process around the electrode pins and the two inserts such that the electrode pins and the inserts are at least partially embedded within the electrode shaft and such that at least one of the electrode pins projects beyond a first end face of the electrode shaft; and
modifying a distance by which the at least one electrode pin projects beyond the first end face.
